(19) United States
(12) Patent Application Publication
(10) Pub. No.: US 2026/0283535 A1
(43) Pub. Date: Sep. 24, 2026

(54) Method and Apparatus for Detecting Consciousness

(71) Applicants: WISCONSIN ALUMNI RESEARCH FOUNDATION, Madison, WI (US); University of Milan, Milan (IT); Tiny Blue Dot, Inc., Santa Monica, CA (US)

(72) Inventors: Giulio Tononi, Verona, WI (US); Christof Koch, Pasadena, CA (US); Marcello Massimini, Milan (IT); Silvia Casarotto, Milan (IT); Michele Angel Colombo, Milan (IT)

(21) Appl. No.: 18/874,366

(22) PCT Filed: Jun. 12, 2023
(86) PCT No.: PCT/US2023/025038

§ 371 Date: Dec. 12, 2024

Related U.S. Application Data
(60) Provisional application No. 63/365,562, filed on May 31, 2022.

(51) Int. Cl. A61B 5/383; A61B 5/00; A61B 5/291; A61B 5/374

(52) U.S. Cl. CPC A61B 5/383; A61B 5/291; A61B 5/374; A61B 5/4821

(57) ABSTRACT

An apparatus to evaluate consciousness processes EEG signals from patient electrodes with a limited spatial coverage. A consciousness index is produced by means of two alternative methods: i) by analyzing time, frequency, and phase relationships in a short window after an electrical or magnetic stimulation of the brain; ii) by analyzing spectral and spatial information, in absence/irrespectively of stimulations.

### CROSS-REFERENCE TO RELATED APPLICATIONS

This application claims the benefit of U.S. provisional application 63/366,562 filed Jun. 17, 2022 and hereby incorporated by reference.

### BACKGROUND OF THE INVENTION

The present invention relates generally to techniques for assessing the presence of consciousness of individuals using electroencephalographic signals collected in absence/irrespectively of stimulations, as well as after non-invasive electrical, magnetic, ultrasonic or other mode of brain stimulation, and in particular to a technique that greatly simplifies and accelerates this assessment.

Disorders of consciousness (DoC) caused by severe brain injury affect more than one million people worldwide each year and are the source of high emotional and financial burden for caregivers, hospitals, and society. Individuals with DoC include coma patients, vegetative state (VS) patients (also known as unresponsive wakefulness syndrome (UWS)), and minimally conscious state (MCS) patients. Another important use case is assessing the presence or absence of consciousness during surgical level anesthesia during any medical procedure.

Demonstrating the presence of covert consciousness is critical for patient assessment and can herald the recovery of behavioral responsiveness. Consciousness is thus a key index that drives prognostic counseling, rehabilitation strategies and even family decisions about withdrawal of life-sustaining therapy (WLST).

At the bedside, a patient's consciousness is typically assessed by the patient's response to command. This behavioral examination, however, may be confounded by motor deficits, contractures, pain, sedation, and/or examiner bias. For this reason, it is desirable to have a measure of consciousness that can be implemented objectively.

One such objective measurement of consciousness is described in U.S. Pat. No. 8,457,731 assigned to the assignee of the present invention and hereby incorporated by reference. This invention exploits an identified link between loss of consciousness and loss of cortical integration caused by decreased communication between otherwise active neurons in different parts of the brain. See generally, Consciousness and Anesthesia, M T Alkire, A G Hudetz, G Tononi, Science, vol. 322. No. 5903, pp. 876-880 (2008) incorporated by reference. Cortical integration can be assessed and quantified by measuring the spatial and temporal spread of neural activity (determined using a multipoint EEG measurement) as a function of time or distance from a point of brain stimulation, for example, from a transcranial magnetic stimulation device.

An alternative approach has been developed which focuses on the complexity of the neural activity after a similar brain stimulation. This measurement produces a “perturbational complexity index” (PCI) by analyzing Lempel-Ziv complexity of the spatial and temporal EEG data. Lempel-Ziv complexity forms the basis for data compression, and in essence this approach uses the degree to which the multipoint EEG signal can be compressed to indicate its complexity.

While both of these measures are promising with respect to providing accurate and objective assessments of consciousness, they can be complex to administer requiring multiple EEG electrodes to obtain the necessary spatial information. In particular, assessment of PCI requires many repeated trials (typically between 100-200) as well as a structural MRI scan to permit accurate off-line reconstruction of brain currents and thus cannot be completed in real time.

### SUMMARY OF THE INVENTION

The present inventors have identified a greatly simplified set of measurements that combine to detect consciousness without requiring large numbers of trials, extensive spatial EEG information, or time-consuming computation and preparation. In the first method, the invention can produce a measure of consciousness by combining simple time domain, frequency domain, and phase quantifications of data taken after stimulation of the brain from a limited number of electrodes and trials, greatly simplifying and accelerating measurements of this type. In a second method, an evaluation of spontaneous (i.e. not-evoked) EEG signals is made to assess the degree of alpha suppression, alpha anteriorization and broadband EEG slowing. These measures can be combined providing a system that eliminates the need for additional brain stimulation steps and equipment.

More specifically, the first invention provides an apparatus for assessing consciousness having an EEG sensor system of a type providing multiple electrodes attached to the skull of the patient to collect electrical signals from active neurons within a patient's brain. A neural stimulator is also provided for triggering a localized brain stimulation of neurons within the patient's brain at a stimulation location and time using electrical, magnetic, or ultrasonic stimulation. An electronic computer receives the electrical signals from the electroencephalographic sensor(s) and executes a stored program to: (i) extract from the electrical signals a first measurement of a time-domain activity in the electrical signals after the time of localized excitation; (ii) extract from the electrical signals a second measurement of a frequency content of the electrical signals after the time of localized excitation; (iii) extract from the electrical signals a third measurement of a time coherence between the electrical signals and the localized excitation of neurons after the time of brain stimulation; and (iv) combine the first, second, and third measurements to provide an output indication of consciousness.

It is thus a feature of at least one embodiment of the invention to detect the likely presence of consciousness in real time and in a wider range of applications, such as patients with disorders of consciousness or during deep anesthesia for medical procedures. Each of the measures can be extracted from a limited set of EEG electrodes and does not require an inverse voltage-to-current source transformation (such as may necessitate separate MRI imaging) and considers cortico-cortical and cortico-subcortical interactions from a causal perspective.

In one embodiment, the first measurement may determine a number of signal peaks in the electrical signals after the time of localized excitation of neurons. The signal peaks may be identified according to a threshold amplitude or threshold derivative.

It is thus a feature of at least one embodiment of the invention to provide a simple method of characterizing an amount of a neuronal “reverberation” after a brain stimulation such as relates to neural integration.

The second measurement may evaluate a difference between high and low spectral energies of the electrical signals after brain stimulation. In one example, the high and low spectral energies may be on either side of 7 Hz.

It is thus a feature of at least one embodiment of the invention to provide a simple assessment of the frequency of cortical responses associated with the presence of consciousness.

The third measurement may determine an inter-trial coherence in electrical signals with respect to the time of repeated brain stimulations.

It is thus a feature of at least one embodiment of the invention to distinguish between causal and noncausal EEG activity using a well-established phase coherence measurement.

The combination of the first, second, and third measurements may provide a weighted sum of the first, second, and third measurements.

It is thus a feature of at least one embodiment of the invention to provide a flexible combination of different metrics that may be adjusted, based on empirical data, by changing the weights.

The first, second, and third measurements may evaluate the electrical signals only in a time window of less than 500 ms after the brain stimulation.

It is thus a feature of at least one embodiment of the invention to provide emphasis to neuronal activity responsive to brain stimulation by a simple windowing of the data with respect to the stimulation.

The output may be such as to exclude measures of electrode location.

It is thus a feature of at least one embodiment of the invention to provide a system that can avoid cumbersome electrode caps with large numbers of electrodes.

Other features and advantages of the invention will become apparent to those skilled in the art upon review of the following detailed description, claims and drawings in which like numerals are used to designate like features.

### BRIEF DESCRIPTION OF THE DRAWINGS

FIG. 1 is a simplified block diagram of a preferred embodiment of the present invention showing a transcranial magnetic stimulation (TMS) coil positionable on top of a patient's head and an electroencephalographic (EEG) system for detecting EEG signals in response to a stimulation, both communicating with a controller executing a stored program;

FIG. 2 is a flowchart showing the steps of the present invention executed at least in part by the controller;

FIG. 3 is a simplified representation of an EEG signal after stimulation showing a peak counting for assessing one measure of the effect of the stimulation;

FIG. 4 is a simplified representation of a power spectrum of the signal of FIG. 3 for assessing high-frequency content associated with neurons not subject to cortical bistability;

FIG. 5 is a simplified representation of two successive acquisitions of EEG signals showing assessment of time coherence with respect to the stimulation;

FIG. 6 is a simplified block diagram of a second preferred embodiment of the present invention showing a collecting of EEG signals without brain stimulation to extract alpha anteriorization, spectral exponent, and alpha power.

FIG. 7 is a plot of power spectral density of EEG signal showing the extraction of 1/f decay and alpha power.

### DETAILED DESCRIPTION OF THE PREFERRED EMBODIMENT

### I. Consciousness Detection Using Brain Stimulation

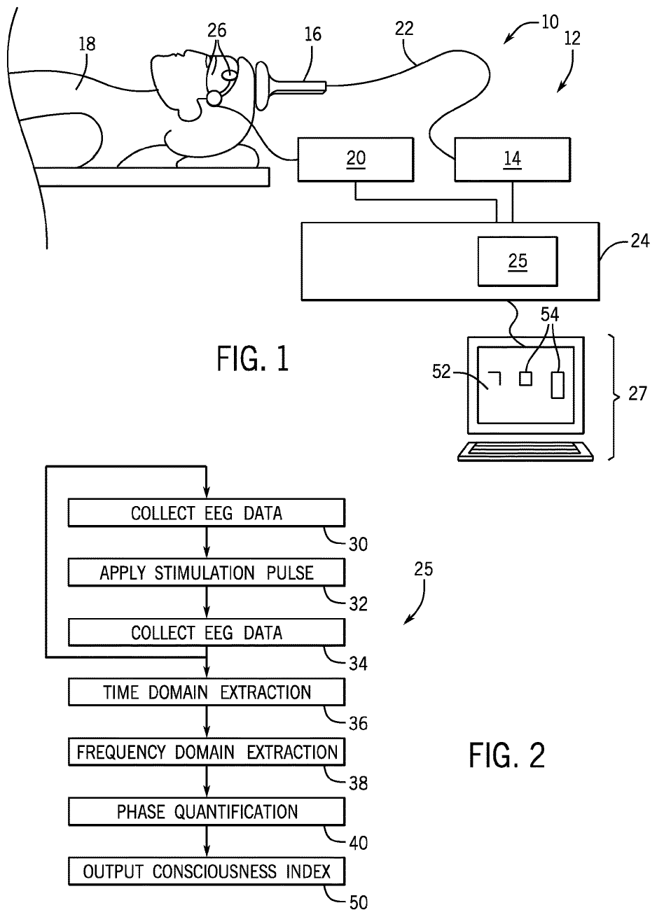

Referring now to FIG. 1, in one embodiment, the apparatus 10 of the present invention may provide a transcranial magnetic stimulation (TMS) device 12 having a power unit 14 and a coil 16 that may be positioned against the head of the patient 18 to provide a localized electrical stimulation to the brain. During operation of the TMS device 12, a set of capacitors or other energy storage devices in the power unit 14 are charged and then rapidly connected to the coil 16 by leads 22 creating a monophasic or biphasic pulse of current in the coil 16 in turn inducing a current within the brain of a patient 18 stimulating neuronal activity. The location of the coil 16 is desirably selected to provide stimulation away from the periphery of the brain and away from any known brain lesions.

In one example, the coil 16 may be a “butterfly coil,” having windings in a figure-eight pattern to provide opposed magnetic flux in adjacent loops, focusing the flux to a compact region. Other coils such as single-loop coils and the like may also be used. TMS devices 12 suitable for use with the present invention are commercially available, for example, from Nexstim of Helsinki Finland under the trade name Nexstim NBS.

The TMS device 12 may be controlled by a controller 24, for example, an electronic computer executing a stored program 25 held in a computer memory, and communicating with a user terminal 27, the latter providing user output and input devices allowing for the output of a consciousness index value, as will be described, and other data, and the input of commands according to methods well known and the art. In this regard, the controller 24 provides interfacing and software to control the timing of the application of the stimulating pulse through the coil 16 or to receive timing data indicating that timing.

The controller 24 may also receive an EEG signals from an EEG processor 20. The EEG processor 20 may provide standard EEG amplifiers, gating circuitry, and filters to provide for continuous EEG signals without disruption by the pulse produced by the TMS device 12. The EEG device is either endowed with a wide dynamic range coupled with a high sampling-rate or sample-and-hold circuits that are specifically developed for use in monitoring EEG during TMS stimulation.

The EEG processor 20 receives signals over leads 21 from a set of cutaneous electrodes 26 placed on the patient's head. As will be discussed in greater detail below, the present invention does not rely on or require spatial identification of the location of each of the EEG signals, and accordingly a high density EEG cap, for example, having in excess of 64 electrodes distributed over the scalp, is believed not to be necessary. The present invention contemplates that fewer than twenty EEG electrodes 26 may be employed and may practically be positioned solely on the patient's forehead and temple in the manner of a standard BIS monitor away from the scalp.

The controller 24 communicates with the EEG processor 20 to receive EEG data for processing indexed to the time of the stimulating signal through the coil 16 as will now be discussed.

Referring now to FIG. 2, during an assessment of consciousness, the controller 24, operating according to the stored program 25, may receive a user command to begin an assessment of the patient 18. At a first step in this assessment, indicated by process block 30, the program 25 provides an instruction to control the EEG processor 20 to collect EEG signals through the electrodes 26, to provide a baseline measurement.

Immediately following this baseline measurement, and as indicated by process block 32, the program may provide a command to the power unit 14 to apply a transcranial magnetic stimulation pulse to the patient 18, as indicated by process block 32.

Following this stimulation pulse, as indicated by process block 34, the controller 24 communicates with the EEG device 20 to collect a second set of EEG signals, the second set exhibiting the effect of the stimulation of process block 32.

The steps of process block 30, 32, and 34 may be repeated more than once for the purpose of establishing EEG coherence as will be discussed below, and more generally may be repeated until a satisfactory signal-to-noise ratio of the measurements is obtained.

Following the acquisition of EEG data of process blocks 30, 32, or 34 (or optionally interleaved with multiple acquisitions) the acquired EEG data is processed to extract three measurements related to time domain behavior, frequency domain behavior, and phase domain behavior of the EEG signals.

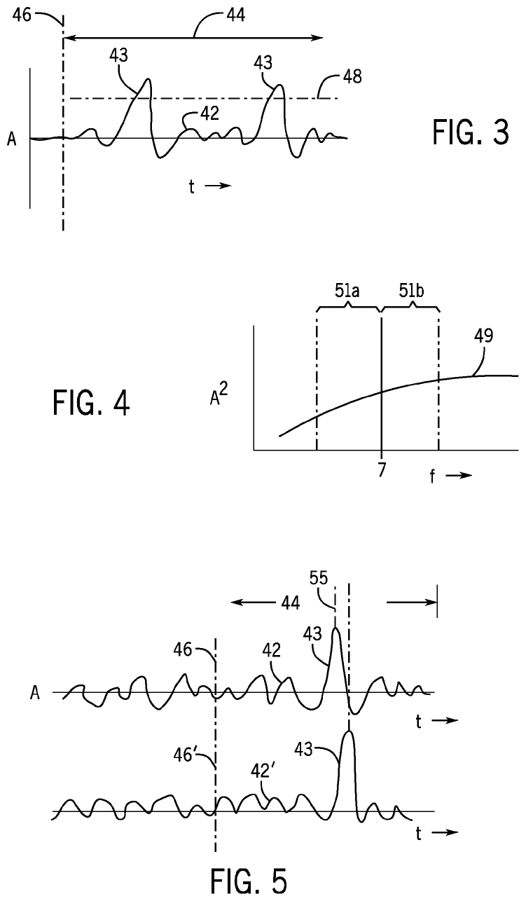

Referring also to FIG. 3, a first time-domain measurement is derived from the EEG signal 42 after the stimulation pulse 46 to indicate reverberatory activity in the cortical and thalamic neurons from the stimulation pulse such as is associated with cortical integration. In one embodiment, this reverberatory activity is assessed by counting peaks 43 in the EEG signal 42 within a predetermined time window 44 after the stimulation pulse 46, for example, 150 ms. The peaks 43 may be defined, for example, according to a threshold amplitude value 48 or as a threshold rate of change (derivative) of EEG amplitude, either or both normalized to average statistics of the EEG signal 42 either before the stimulation pulse 46 or after the time window 44. The peaks 43 may be either positive or negative depending on the particular montage of the measurements made by the EEG processor 20 as will be understood by those of ordinary skill in the art. This measurement process is indicated by process block 36.

The second frequency domain measurement reviews the EEG signal 42, for example, in the same window 44, with respect to its spectral energy, the latter obtained from the power spectrum 49 of the EEG signal 42. This measurement assesses the presence of local maxima above approximately 7 Hz in the spectral profile of the average response. This measurement is indicated by process block 38.

The third phase domain measurement analyzes phase coherence 55 of the EEG signal 42 with the stimulation pulse 46 by reviewing the EEG signal 42, for example, in the same window 44, after multiple different stimulation pulses 46 and 46′ to assess whether the neural activity of the EEG signals 42 is causally related to the stimulation pulses 46. This may be done, for example, by identifying the phase of the EEG signals 42 associated with corresponding peaks 43 in different stimulation pulses 46 and 46′, for example, using the complex Fourier transform, and seeing how much those phases cluster. The clustering of phases can be quantified using inter-trial coherence, a process that adds normalized vectors (phasors) representing each phase together in a vector sum. This measurement may be normalized to a similar measure made of EEG signals 42 outside of the window 44, for example, after the window 44 or before the stimulation pulses 46 or 46′. This measurement is indicated by process block 40.

At process block 50 each of these separate measures of process blocks 36, 38, and 40 may be combined, for example, using a multivariate statistical index aimed at detecting the presence of consciousness. Specifically, a partial least square regression model can be applied on the above-mentioned features to perform multivariate analysis; regression weights can be calibrated on a benchmark dataset where the presence/absence of consciousness can be reliably assessed in communicating subjects based on cither immediate or delayed behavioral reports.

The output of process block 50 may be displayed on the user terminal 27, for example, as a number 52 or in a qualitative way, for example, as a color or bar display 54 or the like

For simplicity and clarity, the invention has been described in the context of a TMS device 12 for stimulation and electroencephalographic cutaneous electrodes for monitoring EEG signals; however, the invention contemplates that the transcranial stimulation by the TMS device 12 and the cutaneous electrodes may be replaced with direct stimulation and detection using intracranial electrodes (not shown).

### II. Consciousness Detection Without Brain Stimulation

This second method of detecting consciousness operates without stimulation of the brain, by evaluating the amount of alpha anteriorization, of broadband EEG slowing, and of alpha suppression; which are respectively quantified as the alpha postero-anterior ratio, the spectral exponent, and alpha power (the latter is divided into an oscillatory and a non-oscillatory component).

### Alpha Anteriorization

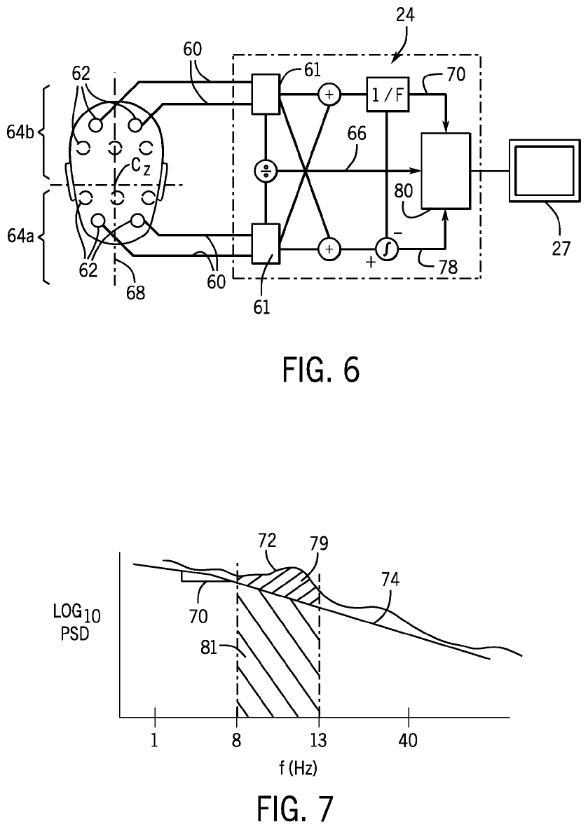

Referring now to FIG. 6, the alpha postero-anterior ratio 66 estimates the amount of alpha anteriorization. For this scope, EEG signals 60 may be collected from a set of scalp electrodes 62 that can be divided into anterior 64a and posterior locations 64b, relative to a line connecting the ears and Cz. The alpha postero-anterior ratio 66 is the ratio of alpha power between electrodes 62 in anterior 64a and posterior 64b locations. Specifically, the method estimates first the geometric mean (represented by processing boxes 61) of both alpha power of electrodes 62 in posterior locations 64b and of alpha power of electrodes 62 in anterior locations 64a; then it determines the postero-anterior ratio of these two means. Values below 1 indicate that anterior alpha activity dominates over posterior activity.

An alternative method to estimate alpha anteriorization, is to estimate the spatial center of mass of alpha power, by weighting the alpha power of each electrode 60 to a parameter proportional to the location of the electrodes 62 along a postero-anterior axis 68. Location values toward the anterior indicate anterior alpha activity dominates over posterior activity.

### Broad-Band EEG Slowing

Referring to FIGS. 6 and 7, the spectral exponent 70 estimates the amount of broad-band EEG slowing, as the steepness of the 1/f-like broad-band decay 74 of the Power Spectral Density background (i.e. the PSD at frequencies where no oscillatory peaks is present 72). The spectral exponent is estimated over a broad range of frequencies (i.e. 1-40 Hz) and averaged across electrodes 62.

More negative values of the spectral exponent 70 indicate steeper spectral decay, hence a larger amplitude ratio of slow-over fast-frequencies, reflecting the slowing of broad-band arrhythmic or quasi-rhythmic activity. More details of this process are described at Palva, S., and Palva, J. M. (2018). Roles of Brain Criticality and Multiscale Oscillations in Temporal Predictions for Sensorimotor Processing. Trends Neurosci. 41, 729-743. doi: 10.1016/J. TINS.2018.08.008 and Colombo, M. A., Napolitani, M., Boly, M., Gosseries, O., Casarotto, S., Rosanova, M., et al. (2019). The spectral exponent of the resting EEG indexes the presence of consciousness during unresponsiveness induced by propofol, xenon, and ketamine. Neuroimage 189, 631-644. doi:10.1016/J. NEUROIMAGE.2019.01.024.both hereby incorporated by reference. The Matlab code is available online (https://github.com/milecombo/spectralExponent/).

### Alpha Suppression

Referring still to FIGS. 6 and 7, alpha power 78 estimates the degree of alpha suppression, and it refers to the narrow-band power spectral density 72 between 8 and 13 Hz. Alpha power 78 is measured separately as two components, the oscillatory and the non-oscillatory component, respectively quantifying the area above 79 and below 81 the 1/f-like trend; such that the two components summed together reflect the total amount of alpha power. The power estimates obtained at each electrode 62 are then averaged together across locations.

Each of the-above mentioned measures are quantitatively combined into a multivariate statistical index aimed at predicting the presence of consciousness, for example, using a partial least square regression model 80, whose regression weights are calibrated on a dataset where the presence/absence of consciousness could be attributed or assessed.

This invention thus provides a joint analysis of a few quantitative neurophysiological features, ncurobiologically grounded and clinically inspired, for the statistical prediction of the presence of consciousness in patients with a DoC. The invention can be extended to other instances of altered states of consciousness (due to pathological, physiological, or pharmacological causes). These neurophysiological features rely on the quantitative analysis of spontaneous (i.e. not-evoked) neurophysiological time series (typically derived from a set of EEG electrodes placed on the scalp), and specifically derive from advanced analysis of their Power Spectral Density.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” or “the microprocessor” and “the processor,” can be understood to include one or more electronic computer processors that can communicate in a stand-alone and/or a distributed environment(s), and can thus be configured to communicate via wired or wireless communications with other processors for devices using standard electrical interfaces and protocols, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

I claim:

1. An apparatus for assessing consciousness comprising:
an EEG sensor providing electrodes at electrode locations to collect electrical signals from firing neurons within a patient's brain;
a neural stimulator for promoting a localized brain stimulation of neurons within the patient's brain at a stimulation location and time using electrical or magnetic stimulation; and
an electronic computer receiving the electrical signals from the EEG sensor and executing a stored program to:
(i) extract from the electrical signals a first measurement of a time domain activity in the electrical signals after the time of localized brain stimulation;
(ii) extract from the electrical signals a second measurement of a frequency content of the electrical signals after the time of localized brain stimulation;
(iii) extract from the electrical signals a third measurement of a time coherence between the electrical signals and the localized brain stimulation of neurons after the time of localized brain stimulation; and
(iv) combine the first, second, and third measurements to provide an output indication of consciousness.

2. The apparatus of claim 1 wherein the first measurement determines the number of signal peaks in the electrical signals after the time of localized brain stimulation of neurons.

3. The apparatus of claim 2 wherein the signal peaks are identified according to a threshold amplitude or threshold derivative.

4. The apparatus of claim 1 wherein the second measurement evaluates the presence of local maxima in the spectral profile of the average response approximately above 7 Hz.

5. The apparatus of claim 1 wherein the third measurement determines an inter-trial coherence in electrical signals associated with multiple localized brain stimulation of neurons.

6. The apparatus of claim 1 wherein the combination of the first, second, and third measurements provides a weighted sum of the first, second, and third measurements.

7. The apparatus of claim 1 wherein the first, second, and third measurements evaluate the electrical signals after the time of localized brain stimulation only in a time window of less than 500 ms after the localized brain stimulation.

8. The apparatus of claim 1 wherein the output does not employ a measure of electrode location.

9. The apparatus of claim 1 wherein the EEG sensor is an electroencephalographic sensor ideally employing less than twenty cutaneous electrodes placed on the patient.

10. The apparatus of claim 1 wherein the neural stimulator is a transcranial magnetic stimulation device.
